(19) United States
(12) Patent Application Publication
(10) Pub. No.: US 2006/0222670 A1
(43) Pub. Date: Oct. 5, 2006

(54) Nanocomposite microgel particles and uses thereof

(76) Inventors: Steven Armes, Sheffield (GB); Syuji Fujii, Sheffield (GB)

Correspondence Address: SHELDON & MAK, INC, 225 SOUTH LAKE AVENUE, 9TH FLOOR, PASADENA, CA, 91101, US

(21) Appl. No.: 11/096,124

(22) Filed: Mar. 31, 2005

(51) Int. Cl. A61K 8/81

(52) U.S. Cl. 424/401; 424/70.17

(57) ABSTRACT

The use of a nanocomposite microgel as a stimulus responsive particulate emulsifier, wherein the microgel particles comprise an inorganic particulate component and a cross-linked responsive polymer matrix.

### BACKGROUND

This invention relates to nanocomposite microgel particles, and more particularly to the use of nanocomposite microgel particles as particulate emulsifiers in oil-in-water and water-in-oil emulsions.

Emulsions find application in a wide range of fields, for example food, agrochemicals, cosmetics, personal/home care product formulations, pharmaceuticals, and tertiary oil recovery. Low molar mass surfactants and surface-active polymers are well known as emulsifiers for use in the preparation of emulsions.

A number of disadvantages are associated with the use of small molecule emulsifiers, however. For example, some are irritants to human skin while the breakdown products of others have been shown to cause damage to the environment. For this reason, the industrial use of alkyl phenol ethoxylate emulsifiers is now being phased out. Emulsions comprising small molecule emulsifiers can be difficult to formulate reproducibly and are relatively unstable to coalescence of the emulsified droplets.

Fine particles are also known to act as emulsifiers. Emulsions stabilized by fine particles are often referred to in the art as “Pickering” or“Ramsden” emulsions. Particulate emulsifiers known in the art include hydrophilic silica sols as emulsifiers for oil-in-water emulsions, and charged latex particles as emulsifiers for water-in-oil emulsions. For a review of particulate emulsifiers see B. P Binks, Curr. Opin. Colloid Interface Sci., 2002, 7, 21 and U.S. Pat. No. 6,428,796, the entire contents of which are hereby incorporated by reference herein.

As particulate emulsifiers are colloidal rather than molecular in nature they may show lower toxicity and reduced irritancy to human skin compared to small molecule emulsifiers. Emulsions produced using particle-based emulsifiers also tend to be more stable to coalescence and the systems in general are more robust, and are therefore easier to make and reproduce.

Emulsions containing small molecule surfactants are prone to foaming as the emulsion is being produced. This can cause significant problems when emulsions are produced on an industrial scale and addition of expensive silicone-based anti-foaming agents is often necessary. Particulate emulsifiers do not usually stabilize foams however and therefore very little or no foam is produced during formulation.

It is known that a significantly higher amount of energy is required to remove particulate emulsifiers from the emulsion interface compared to small molecule emulsifiers. Hence, particle-stabilized emulsions are significantly more stable to breaking (e.g. demulsification) than small molecule-stabilized emulsions.

It is envisaged that the particulate emulsifiers of the present invention may be recovered from the emulsions of the invention and reused. The particles may be recovered by filtration, centrifugation, etc. In contrast, it is difficult to recover small molecule emulsifiers from an emulsion.

It is often desirable to break emulsions once they are formed, for example, to allow release of an agent carried by one of the phases. This can be difficult to achieve in a controlled manner, particularly in the case of particle-stabilized emulsions where the emulsifier is strongly adsorbed at the emulsion interface. So-called demulsifiers can be added to break down emulsions. One disadvantage of this method is that the emulsion cannot be reformed once the demulsifier is added to the system.

Polymeric emulsifiers with stabilizing properties that can be switched on and off are known in the art. Mathur et al. (Nature, 1998, Vol 392, pp 367) have reported soluble copolymers of methacrylic acid and ethylene oxide that stabilize oil-in-water emulsions under acidic conditions when hydrogen bonding interactions cause formation of hydrophobic segments along the polymer chain. An increase in pH disrupts the hydrogen bonding and hence suppresses formation of the hydrophobic sections and leads to the break-up of the emulsion.

Koh et al. (Chem. Commun., 2000, 2461) reported the use of water-soluble poly(N-isopropyl acrylamide)/poly ethylene glycol copolymers for use as thermally responsive emulsifiers. The authors reported reversible gelation of oil-in-water emulsions above the polymer lower critical solution temperature (LCST). However, neither Mathur et al. nor Koh et al. teaches the use of particulate emulsifiers.

There exists a need for non-toxic, non-irritant emulsions that are non-foaming and highly stable to coalescence under normal conditions of use but which can be broken (demulsified) in a controlled manner. In addition, there is a need for emulsions that can be phase inverted in a controlled manner. It is postulated that particulate emulsifiers will be easier and cheaper to synthesize and will give more robust emulsions than soluble emulsifiers.

In WO2004/096422 there is described and claimed the use of a particulate emulsifier comprising at least one polymer, in an oil-in-water or water-in-oil emulsion, wherein the hydrophilic/hydrophobic balance of the polymer can be varied on application of a stimulus to break the emulsion, or to cause phase inversion.

However, there exists a need for improved particulate emulsifiers.

### SUMMARY OF THE INVENTION

In a first aspect, the present invention provides the use of a nanocomposite microgel as a stimulus responsive particulate emulsifier, wherein the microgel particles comprise an inorganic particulate component and a cross-linked responsive polymer matrix.

In a second aspect, the present invention provides a method of manufacturing a stimulus responsive oil-in-water or water-in-oil emulsion wherein there is used a nanocomposite microgel, wherein the microgel particles comprise an inorganic particulate component and a cross-linked responsive polymer matrix, as a particulate emulsifier.

In a third aspect, the present invention provides an oil-in-water or water-in-oil emulsion comprising a nanocomposite microgel, wherein the microgel particles comprise an inorganic particulate component and a cross-linked responsive polymer matrix, as a stimulus responsive particulate emulsifier.

In a fourth aspect, the present invention provides a method of breaking an emulsion, the emulsion comprising a nanocomposite microgel, wherein the microgel particles comprise an inorganic particulate component and a cross-linked responsive polymer matrix, as a stimulus responsive particulate emulsifier, which comprises applying a stimulus to break the emulsion.

In a first preferred embodiment, the invention provides the use of a particulate emulsifier comprising a nanocomposite microgel, wherein the microgel particles comprise an inorganic particulate component and a cross-linked responsive polymer matrix, in an oil-in-water or water-in-oil emulsion, wherein the hydrophilic/hydrophobic balance of the polymeric matrix of the microgel can be varied on application of a stimulus to break the emulsion, or to cause phase inversion. Preferably, the breaking, or phase inversion, of the emulsion is reversible.

In a second preferred embodiment, the invention provides the use of a particulate emulsifier comprising a nanocomposite microgel, wherein the microgel particles comprise an inorganic particulate component and a cross-linked responsive polymer matrix, wherein the stability of the emulsion is dependent on at least one environmental condition.

In a third preferred embodiment, the invention provides oil-in-water and water-in-oil emulsions comprising at least one particulate emulsifier comprising a nanocomposite microgel, wherein the microgel particles comprise an inorganic particulate component and a cross-linked responsive polymer matrix, wherein the hydrophilic/hydrophobic balance of the nanocomposite microgel can be varied on application of a stimulus to break the emulsion, or to cause phase inversion.

In a fourth preferred embodiment, the invention is directed to oil-in-water and water-in-oil emulsions comprising at least one particulate emulsifier comprising a nanocomposite microgel, wherein the microgel particles comprise an inorganic particulate component and a cross-linked responsive polymer matrix, wherein the stability of the emulsion is dependent on at least one environmental condition.

The invention further provides a method for stabilizing an oil-in-water or water-in-oil emulsion comprising the use of a particulate emulsifier comprising a nanocomposite microgel, wherein the microgel particles comprise an inorganic particulate component and a cross-linked responsive polymer matrix, wherein the hydrophilic/hydrophobic balance of the polymeric matrix of the nanocomposite microgel can be varied by application of a stimulus.

In a further aspect, the invention provides a method for preparing an oil-in-water or water-in-oil emulsion, comprising the use of a particulate emulsifier comprising a nanocomposite microgel, wherein the microgel particles comprise an inorganic particulate component and a cross-linked responsive polymer matrix, wherein the stability of the emulsion is dependent on at least one environmental condition.

The invention also provides a method of breaking an emulsion comprising a particulate emulsifier comprising a nanocomposite microgel, wherein the microgel particles comprise an inorganic particulate component and a cross-linked responsive polymer matrix, the method comprising applying a stimulus to vary the hydrophilic/hydrophobic balance of the polymeric matrix of the nanocomposite microgel to an extent sufficient to break the emulsion, or to cause phase inversion.

In a still further aspect, the invention also provides a method of breaking an emulsion comprising a particulate emulsifier comprising a nanocomposite microgel, wherein the microgel particles comprise an inorganic particulate component and a cross-linked responsive polymer matrix, the method comprising varying at least one environmental condition to an extent sufficient to break the emulsion, or to cause phase inversion.

The invention comprises each and every combination of preferred features disclosed herein.

### BRIEF DESCRIPTION OF THE DRAWINGS

FIG. 1 is a schematic representation of pH-induced demulsification using lightly cross-linked poly(4-vinylpyridine)/silica nanocomposites as particulate emulsifiers;

FIG. 2 is a scanning electron micrograph image of lightly cross-linked poly(4-vinylpyridine)/silica nanocomposite particles (dried at pH 6) that were used as particulate emulsifiers;

FIGS. 3a, 3b and 3c are representative optical micrographs of (a) n-dodecane-in-water, (b) methyl myristate-in-water and (c) water-in-1-undecanol emulsions prepared at pH 8;

FIGS. 4a and 4b are (a) transmission and (b) confocal laser scanning images of emulsion droplets prepared at pH 8 using pyrene-labeled fluorescent nanocomposite particles; and

FIGS. 5a, 5b and 5c are digital photographs illustrating the rapid macroscopic phase separation of a methyl myristate-in-water emulsion prepared at pH 8.9 using lightly cross-linked poly(4-vinylpyridine)/silica nanocomposite particles.

### DETAILED DESCRIPTION

The following abbreviations are used herein in reference to monomers (and corresponding polymers).
(P)DMA/(poly)DMA (poly)[2-(dimethylamino)ethyl methacrylate]
(P)MMA/(poly)MMA (poly)(methyl methacrylate]
(P)DEA/(poly)DEA (poly)[2-(diethylamino)ethyl methacrylate]
(P)NIPAM/(poly)NIPAM (poly)(N-isopropylacrylamide)
(P)MEMA/(poly)MEMA (poly)(2-(N-morpholino)ethyl methacrylate]
(P)MAA/(poly)MAA (poly)methacrylic acid
(P)2-VP/(poly)2-VP (poly)2-vinylpyridine
(P)4-VP/(poly)4-VP (poly)4-vinylpyridine
(P)DPA/(poly)DPA (poly)[2-(diisopropylamino)ethyl methacrylate]
(P)TBA/(poly)TBA (poly)[2-(tert-butylamino)ethyl methacrylate]
PPGDA poly(propylene glycol)diacrylate

Some of the above-mentioned monomers can be represented by the following structures:

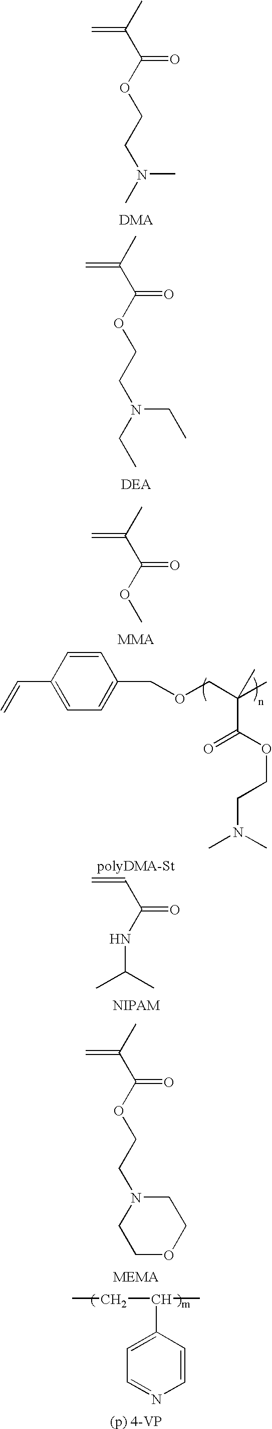

In this specification the term “nanocomposite” refers to particles that have both an organic and an inorganic component and a particle size in the nanosize range, preferably less than 1000 nm.

In this specification, the term “microgels” refers to colloidal particles that are (or can be tuned to become) solvent-swollen.

The nanocomposite microgel particulate emulsifiers of the present invention preferably have a mean particle size of less than about 500 nm, more preferably less than 400 nm, and most preferably less than 250 nm. Preferably, the nanocomposite microgel emulsifiers used according to the invention are in the mean particle size range of from about 1×10⁻⁸ to about 1×10⁻⁵ m, and more preferably from about 5×10⁻⁸ m and to about 5×10⁻⁶ m, for example about 200 nm.

The particles of the nanocomposite microgel particulate emulsifiers used in the present invention preferably have a “currant-bun” or “raspberry” morphology, as described, for example, in M J Percy et al J. Mater. Chem., 2002, 12, 697-702, M J Percy et al Langmuir 2000, 16, 6913-6920, J I Amalvy et al Langmuir 2005 and M Chen et al Macromolecules. 37 (25):9613-9619, 2004, the entire contents of which are hereby incorporated herein by reference. The “currant-bun” morphology, wherein inorganic particles are substantially uniformly distributed throughout the nanocomposite microgel particles, is particularly preferred.

A nanocomposite microgel particle preferably comprises a plurality of inorganic particles embedded in a cross-linked responsive polymer matrix. Preferably at least some of the inorganic particles are on the outer surface of the microgel particle. The inorganic particulate component can be chosen from a wide range of suitable materials, but preferably is substantially water insoluble and preferably substantially inert. Preferred inorganic components can, however, interact with the responsive polymer matrix as will be described hereinafter. Suitable inorganic materials include, for example, inorganic oxides and hydrated oxides, and inorganic salts. Preferred examples include silica, alumina, Fe₂O₃, BaSO₄, CaSO₄ and CaCO₃ sols.

Preferably, the inorganic particulate component comprises silica particles. Preferred silica particles for use in the present invention include those derived from aqueous silica sols having a mean particle size of from about 1 nm to about 100 nm, preferably from 10 nm to 30 nm, and a concentration of from about 1 to about 60 w/w %, preferably from 20 to 60 w/w %. Alumina-coated silica particles can also advantageously be used.

The volume fraction of inorganic particulate component in the microgel particles is preferably from about 0.1 to about 0.5, and more preferably from about 0.2 to about 0.4, for example, about 0.33.

An emulsion is a mixture of immiscible liquids wherein one liquid is finely dispersed within the continuous phase of another. Emulsions are generally characterised as oil-in-water emulsions, wherein oil droplets are dispersed in a continuous water phase, or water-in-oil emulsions, wherein water droplets are dispersed in a continuous oil phase.

The nanocomposite microgel emulsifiers used according to the present invention are particles that have variable hydrophilicity or hydrophobicity. In other words, the affinity for water (or, conversely, oil) of the particles can be varied by varying at least one environmental condition. The stability of emulsions comprising such particles is therefore dependent on the environmental conditions.

Particles are classed as hydrophilic if the contact angle they make with an oil-water interface (measured into the water phase) is less than 90 degrees. Particles are classed as hydrophobic if this contact angle is greater than 90 degrees.

Hydrophilic particles are more hydrophilic if the contact angle approaches zero. Hydrophobic particles are more hydrophobic if the contact angle approaches 180 degrees.

Without wishing to be bound by any particular theory, it is postulated that in order to form stable emulsions the nanocomposite microgel emulsifiers used according to the invention should have some affinity for both the oil and water phases of the emulsion. When the particles have affinity for both phases of the emulsion, the particles adsorb at the oil/water interface of the emulsion forming an adsorbed layer around the dispersed phase that prevents coalescence of the dispersed phase and therefore stabilizes the emulsion. When the particles do not have affinity for both of the phases in the emulsion, or have significantly stronger affinity for one of the phases, the particles do not adsorb at the emulsion interface and so do not form a stabilizing layer. A stable emulsion in which the particles have affinity for both phases can therefore be broken by changing the hydrophilic/hydrophobic balance of the particle emulsifier such that the particle no longer has affinity for both phases of the emulsion, or has a much stronger affinity for one phase of the emulsion relative to the other phase.

In this specification, a stable emulsion is considered to be “broken” on the appearance of a macrophase caused by coalescence of the particles of the disperse phase.

Phase inversion occurs when an oil-in-water emulsion is converted into a water-in-oil emulsion; or vice versa. Phase inversion can occur directly, without the intermediate formation of a macrophase.

The nanocomposite microgel emulsifiers used according to the invention should remain essentially intact in either phase of the emulsion, that is, they should not completely disintegrate in either phase.

The nanocomposite microgel emulsifiers used in the present invention comprise at least one cross-linked polymer matrix that displays variable hydrophilicity/hydrophobicity depending on external conditions. The term “responsive polymer” is used herein to describe any polymer whose hydrophilic/hydrophobic balance can be changed by varying one or more external condition(s) and the term “stimulus responsive particulate emulsifier” is used to describe a particulate emulsifier wherein the hydrophilic/hydrophobic balance of the surfaces of the particles can be changed by varying one or more external conditions.

In a good solvent the polymer chains that form the matrix of the microgel particles are solvated, which means that solvent enters the microgel particle and the particle is swollen. Even when fully solvated, the cross-links mean that the microgel particle retains its particle structure and does not completely dissolve or disintegrate in the solvent. In poor solvents, solvent is excluded from the microgel particle interior and the microgel forms a collapsed compact structure.

The microgels used in this invention comprise a cross-linked polymer matrix wherein at least some of the polymer chains forming the matrix comprise a responsive polymer as previously defined. They can be stabilized in suspension by any means known in the art for stabilizing suspended solid particles, for example electrical double layer (charge or surfactant) stabilization or steric (polymeric) stabilization.

The polymer matrix of the microgel particles can be attached to the particulate component by any means as long as the attachment to the particulate component is sufficiently strong such that little or no disintegration of the microgel particles occurs when they are in use as emulsifiers. The matrix can be attached by physical (e.g. hydrogen bonds, hydrophobic or acid-base interactions) or chemical (e.g. covalent or ionic bonds) means. One preferred type of microgel according to the invention is a charge-stabilized microgel formed from a cross-linked responsive polymer matrix.

An alternative preferred nanocomposite microgel structure comprises a cross-linked responsive polymer matrix that is sterically-stabilized. Preferably, steric stabilization is provided by polymer chains that are different than those forming the microgel matrix. In one particularly preferred embodiment, the steric stabilizing layer also comprises a responsive polymer.

The particles of the invention may be formed from one or more responsive polymer(s). Mixtures of responsive and non-responsive polymers may also be used to form the polymer matrix. A responsive polymer can also be a polymer formed from comonomers having responsive and non-responsive moieties, provided that the responsive moiety is present in an amount sufficient to provide the appropriate response under an applied external condition/stimulus. The total amount of responsive polymer in the nanocomposite microgel emulsifier particles must be sufficient to make the particles responsive to at least one external condition/stimulus.

The polymer may respond to a variation of an external condition by, for example, a steric or conformational change, by a change in protonation or de-protonation, by a change in salvation, by a change in hydration, or by any other response leading to a change in the contact angle of the microgel particle surface with an oil-water interface. Preferably the change in contact angle is at least about 10°, more preferably at least 30°, even more preferably up to 50° and most preferably up to 70°.

For many applications the requirement is to break the emulsion, or to cause phase inversion, quickly, and preferably as quickly as possible. In preferred embodiments of the invention, the emulsion is broken, or phase inversion takes place, in less than about 1 hour, more preferably less than 15 minutes, even more preferably less than 5 minutes, and most preferably less than 1 minute.

The stability of emulsions that are produced using nanocomposite microgel particulate emulsifiers comprising responsive polymers in accordance with the invention is dependent on the external environmental conditions. Varying the environmental conditions therefore affects the stability of the emulsion. Any stimulus which is capable of changing one or more environmental condition(s) and hence the hydrophilic/hydrophobic balance of the responsive polymer can be used to break, or cause phase inversion of, the particle-stabilized emulsions according to the invention.

Environmental conditions that may affect the hydrophilic/hydrophobic balance of the responsive polymer include pH, temperature and ionic strength. Preferred stimuli according to the invention therefore include a change in temperature, a change in pH or a change in ionic strength, for example, due to the addition of a suitable salt/electrolyte.

The degree and nature of the sensitivity of the particulate emulsifiers to the external condition(s) can be tailored by varying the nature and/or amount of the responsive polymers. For example, by selecting a pH-responsive polymer, it is possible to produce an emulsion the stability of which is pH-dependent.

A responsive polymer may be responsive to more than one stimulus. For example, the hydrophilic/hydrophobic balance of a polymer may depend on both the pH and the temperature of its environment. An emulsifier that is sensitive to more than one external condition may therefore be prepared by utilizing such a polymer. Alternatively, two or more different responsive polymers may be used, each of which is responsive to a different external stimulus.

Demulsification, or phase inversion, of an emulsion according to the invention may be reversed by reversing the environmental change used to produce demulsification or phase inversion. A change in emulsion pH or temperature, for example, is reversible. Some of the stable emulsions according to the invention break down, or invert, when the pH of the emulsion is lowered. However, if the pH of the broken or inverted emulsion is raised to above the pH at which demulsification or inversion occurred, a stable emulsion may reform. Similarly, demulsification or inversion produced by raising or lowering the emulsion temperature may be reversed by lowering or raising the temperature, respectively.

The choice of responsive polymer for use in the particulate emulsifiers of the invention depends inter alia on which particular stimulus is required to break the emulsion, or cause phase inversion, and on the nature and intended use of the emulsion. Properties of the bulk polymer can be used as a guide for selecting which responsive polymers would be suitable for use in the present invention. For example, any polymer which has affinity for oil and water at pH values on one side of its pKa value but only affinity for water on the other side is potentially suitable for use as the responsive polymer matrix in the particulate emulsifiers for use according to the invention.

The responsive polymer used according to the present invention can be any suitable polymer that has variable hydrophilicity. In other words, any polymer that changes its affinity for water (and conversely, oil) in response to an external stimulus or changing environmental condition(s) may be suitable.

The responsive polymer is not limited by way of polymer architecture. The responsive polymer may therefore comprise a homopolymer or copolymers such as statistical, alternating, graft, star and block copolymers. The responsive polymer may also comprise any number of different comonomers.

Preferably, the responsive polymer matrix comprises a vinyl polymer microgel, for example, a polyvinylpyridine, poly(meth)acrylate (preferably a tertiary amine-substituted (meth)acrylate), or poly(meth)acrylamide. Most preferably, the polymer matrix comprises cross-linked poly4-VP, poly2-VP, polyDEA, polyDMA, polyMEMA, or polyDPA.

Preferably, the particulate emulsifier is a cross-linked vinyl polymer-silica nanocomposite microgel. In a most preferred embodiment, the microgel particles comprise silica particles embedded in a cross-linked poly4-VP matrix with poly4-VP chains anchored to the surface of the silica particles. It is believed that the interaction between the silica particles and the poly4-VP matrix plays a significant part in stabilizing the nanocomposite microgel particles during the polymerization reaction.

Preferred responsive polymers for use according to the invention are polymers that display a change in hydrophilic/hydrophobic balance in response to a change in pH, temperature or ionic strength. Polymers that show pH, temperature, or ionic strength sensitivity are well known in the art (see for example, Angew. Chem. Int. Ed., 2001, 40, No. 12, 2328-31; Angew. Chem. Int. Ed., 2002, 41, No. 8, 1413-16; J. Am. Chem. Soc., 1998, 120, 12135-12136; J. Am. Chem. Soc., 2001, 123, 9910-9911; Polymer, 42 (2001), 5993-6008; Macromolecules 1999, 32, 2088-2090; Chem. Commun., 2002, 2122-2123; and Current Opinion in Colloid & Interface Science 6 (2001) 249-256), and references cited therein, the entire contents of which are incorporated herein by reference.

pH Responsive Polymers

Polymers that have variable hydrophilicity depending on pH generally include any polymers comprising acidic or basic functional groups on the polymer chain. The degree of hydrophilicity of such polymers depends on the degree of protonation or deprotonation of such functional groups. Preferably, the functional groups are either weakly acidic or weakly basic. The responsive polymers suitable for use according to this aspect of the invention are preferably responsive between the range of pH 2 to pH 12, more preferably pH 3 to pH 10. Suitable functional groups include amines and carboxylic acids. Many such polymers are known in the art and include, for example, polymers prepared from (meth)acrylic acid monomers and derivatives thereof. Particular examples include polyDMA, polyMEMA, polyDEA, polyDPA, polyTBA, polyAA and polyMAA. Other preferred polymers include poly4-VP and copolymers of 4-VP, poly2-VP and poly N-vinylimidazole. Especially good results have been obtained with poly4-VP and copolymers of 4-VP, which are the preferred polymers for use in the present invention.

Polymers comprising acidic or basic residues display different hydrophilicity above and below the polymer (or conjugate acid) pKa value. Poly4-VP and copolymers of 4-VP, poly2-VP and poly N-vinylimidazole, for example, all have a ring nitrogen atom and are substantially water-insoluble above the pKa of the conjugate acid when the nitrogen atoms are not protonated, but are water soluble below the pKa when the nitrogen-containing ring is protonated and most nitrogen atoms carry a positive charge. PolyDEA and polyDPA, for example, which include a tertiary amine group on each monomer residue, are substantially water-insoluble above the pKa of the conjugate acid when the amine groups are not protonated, but are highly water soluble below the pKa when the amine groups are protonated and most groups carry a positive charge. PolyDMA and polyMEMA, for example, which also include a tertiary amine group on each monomer residue, are water-insoluble above the pKa of the conjugate acid when the amine residues are not protonated but the hydrophilicity is significantly increased when the amine groups are protonated below the polymer pKa.

The stimulus necessary to change the hydrophilicity of such polymers is therefore a change in pH. The pKa value of a polymer or conjugate acid of a polymer indicates the pH at which 50% of the acid groups are deprotonated. Any polymer which has an acidic or basic moiety on each monomer residue comprises a large number of acidic or basic groups. The change in hydrophilicity of such polymers is not necessarily therefore a sharp transition around the polymer pKa as the pH is adjusted around the pKa value. Complete protonation or deprotonation may therefore take place at pH values well above or below the pKa. The number and nature of the acidic and/or basic functional groups can be varied to vary the degree of pH responsiveness and the pH value at which breaking or inversion of a particular emulsion occurs.

The choice of pH-responsive polymer to incorporate into the emulsifier particle used according to the invention depends on the pH at which the change in hydrophilicity is required. In the case of poly4-VP, either oil-in-water or water-in-oil emulsions can be prepared, depending on the polarity of the oil phase, at around pH 8-9. Rapid demulsification occurs below pH 3, since the addition of acid leads to the protonation of the 4-VP residues, which in turn imparts hydrophilic character to the nanocomposite microgel particles, hence promoting their desorption.

In the case of polyDMA, for example, the polymer is weakly hydrophilic above the pKa of the conjugate acid when the amine groups are less protonated (around pH 8) and also has affinity for a number of oils such as toluene and (hot) n-hexane. Hence, this polymer adsorbs at the oil/water interface for a number of oil and water combinations. Particles comprising this polymer are therefore able to form stable emulsions above the pKa of the polymer conjugate acid. Below the pKa when the polymer is fully protonated, however, the polymer has no affinity for oil and does not therefore adsorb at the oil/water interface of an emulsion. Hence, a stable emulsion comprising this polymer at pH 8 can be broken by lowering the pH to the point where the polymer chain is highly protonated (typically at or below about pH 4).

Temperature Responsive Polymers

Many water-soluble polymers show inverse temperature solubility behaviour in aqueous solution. This means that if a solution of the polymer in water is heated, the polymer becomes less soluble due to a decrease in hydrogen bonding between the polymer and the solvent.

Water-soluble polymers having inverse temperature solubility behaviour display a lower critical solution temperature (LCST) in aqueous solution. Below the LCST the polymer is hydrophilic, but above the LCST the polymer becomes insoluble in water and phase separation occurs. Hence, the hydrophilic/hydrophobic balance of the polymer changes around the LCST. The LCST depends on inter alia the hydrophilicity of the polymer and the polymer molecular weight.

Very hydrophilic polymers have a high LCST. For example, the LCST of polyMEMA is around 40-50° C. in the absence of any added salt. Polymers that are less hydrophilic have a lower LCST. For example, the LCST of polyDMA is around 35 to 45° C. depending on its molecular weight. Some polymers that are not usually considered to be hydrophilic because they are insoluble in water at room temperature, may have an LCST below room temperature and so may become soluble on cooling to below room temperature (e.g. poly(propylene oxide), which has an LCST of around 15° C.). Some polymers have a very sharp transition from water-soluble to water-insoluble as the solution is heated above the polymer LCST, for example poly(N-isopropylacrylamide) which has an LCST of 32° C. in pure water.

The responsive polymer may comprise a copolymer comprising a combination of monomers that display inverse temperature dependent solubility. Copolymer compositions can be tailored to provide a polymer that has a particular preferred LCST. For example, a copolymer of A and B would have an LCST that lies between the LCSTs of homopolymers comprising only A or only B.

When incorporated into the particles used according to the invention, the above polymers provide particles with the ability to change hydrophilicity depending on external temperature. Polymers that are only weakly hydrophilic at room temperature have some affinity for both the oil and water interfaces of emulsions. Particles comprising such polymers therefore stabilize oil-in-water emulsions at this temperature. When the emulsion is heated above the polymer LCST, however, the responsive polymer, and hence particle, no longer has affinity for the water phase and the emulsion therefore breaks, or inverts.

Ionic Strength

The water solubility of a polymer can also be changed by changing the ionic strength of an aqueous solution of the polymer, for example, by adding salt to the aqueous solution, or by diluting or concentrating an aqueous salt solution of the polymer. A number of water-soluble polymers are known to precipitate out of solution when salt is added. Ions from the salt compete for the polymer's hydration layer of water molecules, which means that salvation of the polymer is reduced leading to the polymer precipitating out of solution. Hence, salt can be used to adjust the water solubility of polymers and can therefore be used as a trigger to break emulsions according to the present invention comprising salt-sensitive polymers.

Water-soluble polymers known to precipitate out of solution on the addition of salt include, for example, poly(ethylene oxide), polyDMA and polyMEMA. The minimum amount of salt required varies according to the nature of the polymer, with polyMEMA being particularly sensitive to ‘salting-out’. Another factor that can play a role is the solution pH: a lower solution pH can require extra salt to cause precipitation, or even prevent precipitation.

The cross-link density of the responsive polymer matrix is preferably relatively low in order to allow for swelling of the polymer by ingress of liquid, for example, water. Preferably the cross-link density is less than about 10%, more preferably less than about 5% and most preferably less than about 3%. For many of the nanocomposite microgels of the invention the cross-link density of the responsive polymer is within the range of from about 0.1% to about 5%, and more preferably from about 0.5% to about 3.0%.

Cross-linking can be accomplished in a variety of ways, including the use of ionizing radiation and free radical generating chemical cross-linking agents. Preferably the responsive polymer is prepared by polymerizing the appropriate monomer or monomers in the presence of a cross-linking agent, preferably a di-functional monomer that can react with the polymerizing monomer and cross-link the polymer chains. Preferred cross-linking agents include, for example, ethylene glycol dimethacrylate [EGDMA], polypropylene glycol diacrylate or divinyl benzene. The quantity of cross-linking agent present in the responsive polymer is preferably in the range from about 0.1 mole % to about 5 mole %, more preferably from about 0.5 mole % to about 3 mole %.

The emulsions produced according to the invention can be oil-in-water or water-in-oil emulsions. The choice of particulate stabilizer depends on which type of emulsion is to be formed, on the nature of the two phases and on the intended use of the emulsion. Oil-in-water emulsions have gained increasing commercial importance over recent years. Hence, in a preferred embodiment of the present invention, the particulate emulsifiers are used to provide stable oil-in-water emulsions that can be controllably, and preferably reversibly, broken or inverted on application of an external stimulus.

The emulsions according to the invention may be used in, for example, foods, agrochemicals, cosmetics, personal/home care product formulations, and pharmaceuticals. For example, an oil-in-water emulsion according to the invention may be designed to break at the surface pH of the human skin, at physiological temperatures and/or upon dilution of the emulsion, such that the oil phase or an ingredient carried by the oil phase is then deposited on contact with (wet) skin or hair. Such emulsions could be used in, for example, shampoos or moisturising compositions, or to deliver active ingredients to the skin.

The emulsions of the invention may be useful in pharmaceutical formulations, including topical formulations and formulations for oral drug delivery. For example, an emulsion that breaks at the pH of the human stomach could be used to deliver an active ingredient carried in the dispersed phase to the stomach. Alternatively, the dispersed phase of an emulsion which is stable at the pH of the human stomach, but designed to break at the higher pH of the intestines could be used to carry an active ingredient through the stomach for delivery to the intestines. Such emulsions may be particularly suitable for use in the oral delivery of acid-sensitive or acid-labile active ingredients through the stomach to the intestinal tract.

The droplet size of the emulsions according to the invention is preferably in the range of from about 0.3 to about 100 μm. In general, the smaller the size of the particulate emulsifier, the smaller the droplet size in the emulsion. Other factors that determine the final emulsion droplet size include the energy (e.g. stirring rate, time) imparted in generating the emulsion and the concentration of the particulate emulsifier.

The emulsions of the invention may comprise additional components in either the oil or the water phase. Additional components are not particularly limited provided that they do not prevent formation of the emulsions or adversely affect emulsion stability. Examples of additional components include, for example, food additives, flavourings, agrochemicals, pharmacologically active ingredients, or cosmetic ingredients (e.g. fragrances) to be delivered to the skin or hair.

The following non-limiting Example further illustrates the invention.

### EXAMPLE

This Example describes the preparation of cross-linked poly(4-vinylpyridine)-silica nanocomposite particles by copolymerizing 4-vinylpyridine [4VP] with ethylene glycol dimethacrylate [EGDMA] in the presence of an ultrafine aqueous silica sol.

Experimental Materials

The three oils used in this example, n-docdecane, methyl myristate and 1-undecanol, each had purities of at least 99%. The 4-VP and EGDMA crosslinker were each treated in turn with basic alumina in order to remove inhibitor and stored at −20° C. before use. The ultrafine silica sol (SiO₂) was Nyacol 2040 (supplied as a 40 w/w % aqueous dispersion by Eka Chemicals, Sweden; these particles have a mean diameter of around 20 nm and possess a negative surface charge with sodium counter-ions). The free radical initiator used for the copolymerizations was ammonium persulfate.

Nanocomposite Syntheses

The nanocomposite particles were prepared by free radical copolymerization of 4-VP and EGDMA in the presence of 20 nm silica sols in aqueous media as follows. An aqueous solution of the Nyacol 2040 silica sol (19.3 ml; equivalent to 8.0 g of dry weight silica) was mixed with 80 ml de-ionized water, 4 VP (10.0 g) and EGDMA (0.10 g) in a three-necked round bottom flask; two necks were sealed with a suba seal and the third neck was connected to a condenser. This set-up allowed both N₂ inlet and addition of initiator using syringe needles. The mixture was purged with nitrogen and placed in an oil bath maintained at 60° C. equipped with a magnetic stir bar. Ammonium persulfate initiator (0.10 g; 1.0 wt. % based on monomer) was dissolved in water (8.7 mL), separately purged with N₂ and added to the reaction vessel. The reaction mixture was stirred at 60° C. for 24 h.

A non-crosslinked nanocomposite particle synthesis was carried out in the absence of any EGDMA using the same protocol as that described by M. J. Percy et al Langmuir, 2000, 16, 6913, the entire contents of which are hereby incorporated herein by reference. The two milky-white dispersions were purified by at least three centrifugation-redispersion cycles, with each successive supernatant being decanted and replaced with doubly distilled water until no excess silica sol was observed by transmission electron microscopy. Care was taken to avoid excessive centrifugation rates and sedimentation times, since these would otherwise result in the unwanted sedimentation of the excess silica sol and also make redispersion of the nanocomposite particles more difficult. A pyrene-labelled nanocomposite was synthesized under the same conditions as the conventional nanocomposites described above using both 1-pyrenylmethyl methacrylate [8] and EGDMA as comonomers (each added at 1.0 wt. % based on 4-vinylpyridine, respectively).

Nanocomposite Characterization

Dynamic light scattering (DLS) studies. Measurements were made at 20° C. using a Brookhaven Instruments Corp. BI-200SM goniometer equipped with a BI-9000AT digital correlator using a solid-state laser (125 mW, λ=532 nm) at a fixed scattering angle of 90°. Intensity-average particle diameters (Dz) and polydispersities were calculated by cumulants analysis of the experimental correlation function using the Stokes-Einstein equation for dilute, non-interacting monodisperse spheres.

Scanning Electron Microscopy. SEM studies were carried out using a Leo Stereoscan 420 instrument operating at 20 kV and 5-10 pA. Dried samples were placed on an aluminium stub and sputter-coated with gold to minimise sample-charging problems.

Thermogravimetric analysis. Thermogravimetric analyses were carried out using a Perkin-Elmer TGA-7 instrument. The nanocomposite particles were heated from 20° C. to 800° C. at a scanning rate of 20° C. per minute under air. The organic copolymer was completely pyrolyzed under these conditions but the silica remained thermally stable. After correction for the water content of the hydrated silica sol under ambient conditions, the observed mass losses corresponded to the silica contents of the nanocomposite particles.

Preparation of Emulsions

Stock solutions (1.0 wt. % solids) of the nanocomposite dispersions were prepared by serial dilution after centrifugal clean-up. The solution pH of 5.0 mL aliquots of these diluted dispersions was monitored using a pH meter and adjusted as required by adding a few drops of either concentrated HCl or NaOH. These aqueous dispersions were then homogenized with 5.0 mL of either n-dodecane, methyl myristate or 1-undecanol for two minutes at 20° C. using an IKA Ultra-Turrax T-18 homogenizer with a 10 mm dispersing tool operating at 12,000 rpm. Emulsion stabilities after standing for 24 h at 20° C. were assessed by visual inspection.

Emulsion Characterization

Conductivity Measurements. The conductivity of the emulsions immediately after preparation was measured using a digital conductivity meter (Hanna model Primo 5). The conductivities of aqueous nanocomposite dispersion ranged from 40 to over 2,000 μS cm⁻¹, depending on the solution pH. The emulsions were classified according to their conductivities. A high conductivity indicated an oil-in-water emulsion and a low (<1 μS cm⁻¹) conductivity indicated a water-in-oil emulsion.

Optical Microscopy (OM). A drop of the diluted emulsion was placed on a microscope slide and viewed using an optical microscope (James Swift MP3502, Prior Scientific Instruments Ltd.) fitted with a digital camera. This technique was used to estimate the mean droplet diameter.

Malvern MasterSizer. A Malvern MasterSizer S instrument equipped with a small volume (ca. 15 mL) MS-7 magnetically stirred cell and a 2 mW HeNe laser operating at 633 nm was used to size the emulsions. A 300 F lens was used, which enabled droplet diameters from 0.50 to 900 μm to be measured. The diluted emulsion was placed in the cell using a plastic pipette. The stirring rate was adjusted to avoid creaming of the emulsion. Corrections were made for background electrical noise and laser scattering due to contaminants on the optics and within the sample. Each emulsion was analyzed ten times and the best five runs were selected for averaging. Typical analysis times were two minutes per sample after alignment and background measurements. The raw data were analyzed using Malvern software. The mean droplet diameter was taken to be the volume mean diameter (D4/3), which is mathematically expressed as D4/3=Σ Di4Ni/Σ Di3Ni. The standard deviation for each diameter provides an indication of the size distribution. After each measurement, the cell was rinsed three times with ethanol followed by three times with doubly-distilled water. The glass walls of the cell were carefully wiped with lens cleaning tissue to avoid cross-contamination and the laser was aligned centrally on the detector.

Confocal Laser Scanning Microscopy. A confocal laser scanning microscope (Zeiss LSM 510 Meta on a Axiovert 200M microscope) was used for the confocal fluorescence and transmission microscopy studies. The microscope was equipped with argon ion uv and HeNe gas lasers, operating at wavelengths of 351 nm (for excitation of the pyrene groups in the nanocomposite particles) and 543 nm, respectively. The diluted emulsion was placed on the glass slide and a cover glass was placed over it. The droplets were observed using a water immersion laser technique. Transmission images were obtained at a laser wavelength of 543 nm and confocal laser scanning images were produced using an emission wavelength greater than 385 nm. Two scans in an XY array were averaged to produce the images.

Results

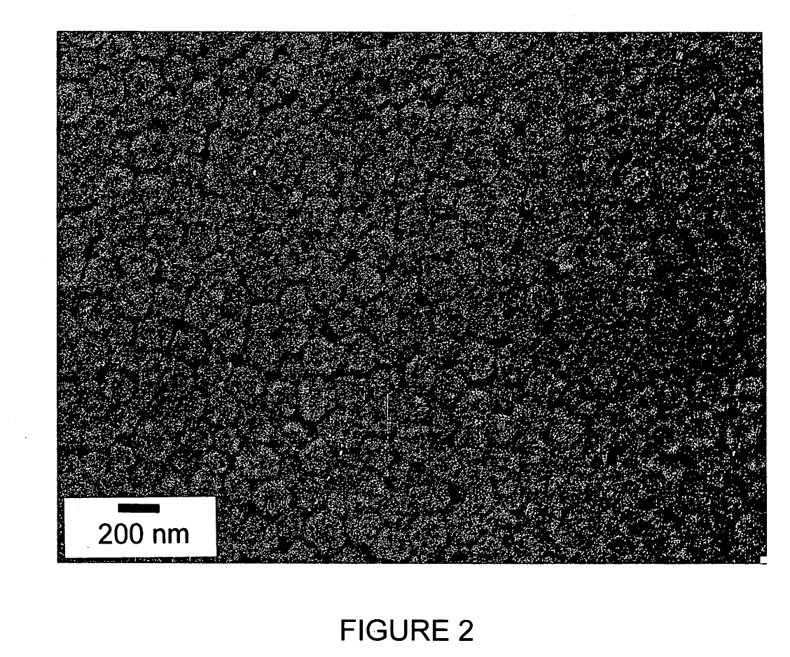

Scanning electron microscopy studies of the dried, lightly-crosslinked P4VP/SiO₂ nanocomposite particles (see FIG. 2) indicated an average particle diameter of around 200 nm. Thermogravimetric analysis gave a mean silica content of approximately 35%, which is consistent with the silica content reported by Percy et al Langmuir, 2000, 16, 6913. Electrophoresis studies on dilute aqueous dispersions of the lightly crosslinked P4VP/SiO₂ particles indicated an isoelectric point at approximately pH 6.

Dynamic light scattering studies of highly dilute aqueous dispersions of the P4VP/SiO₂ nanocomposite particles with varying solution pH confirmed that (i) their intensity-average diameter at pH 8.8 was approximately 230 nm and (ii) significant swelling occurred below pH 4 due to protonation of the 4-vinylpyridine residues. Crosslinking is essential, since particle dissolution was observed at low pH for non-crosslinked P4VP-SiO₂ nanocomposites. At pH 2.5 the lightly cross-linked particles are protonated and acquire significant cationic microgel character; their intensity-average particle diameter is around 550 nm, indicating more than an order of magnitude increase in volume.

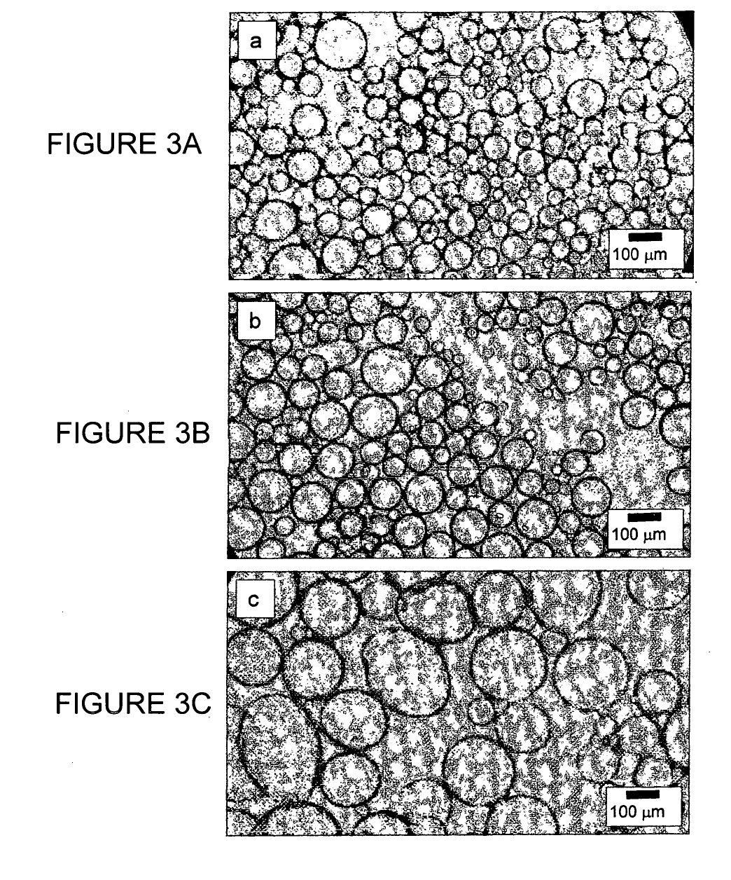

1% aqueous dispersions of these lightly cross-linked P4VP/SiO₂ nanocomposite particles proved to be effective particulate emulsifiers for each of the three oils used in this study, namely n-dodecane, methyl myristate and 1-undecanol, see Table 1. Electrical conductivity measurements and droplet tests indicated that oil-in-water emulsions were obtained at pH 8-9 for the first two oils, while a water-in-oil emulsion was obtained with the more polar 1-undecanol under the same conditions. Typical optical microscopy images of these three emulsions are shown in FIGS. 3a, 3b and 3c. As shown in FIGS. 3a and 3b, the n-dodecane-in-water and methyl myristate-in-water emulsion droplets were spherical and fairly polydisperse, ranging from 20 to 100 μm. As shown in FIG. 3c, the water-in-1-undecanol emulsion droplets were less spherical and more polydisperse, ranging from 20 to 350 μm. The volume-average size distributions were estimated using the Malvern MasterSizer instrument (see Table 1). Each emulsion in FIGS. 3a, 3b and 3c was prepared at 20° C. using 50% oil and 50% aqueous solution containing 1 wt. % poly(4-vinylpyridine)/silica nanocomposite particles (1.0 wt. % crosslinked) as an emulsifier. The solutions were pH adjusted using aqueous NaOH.

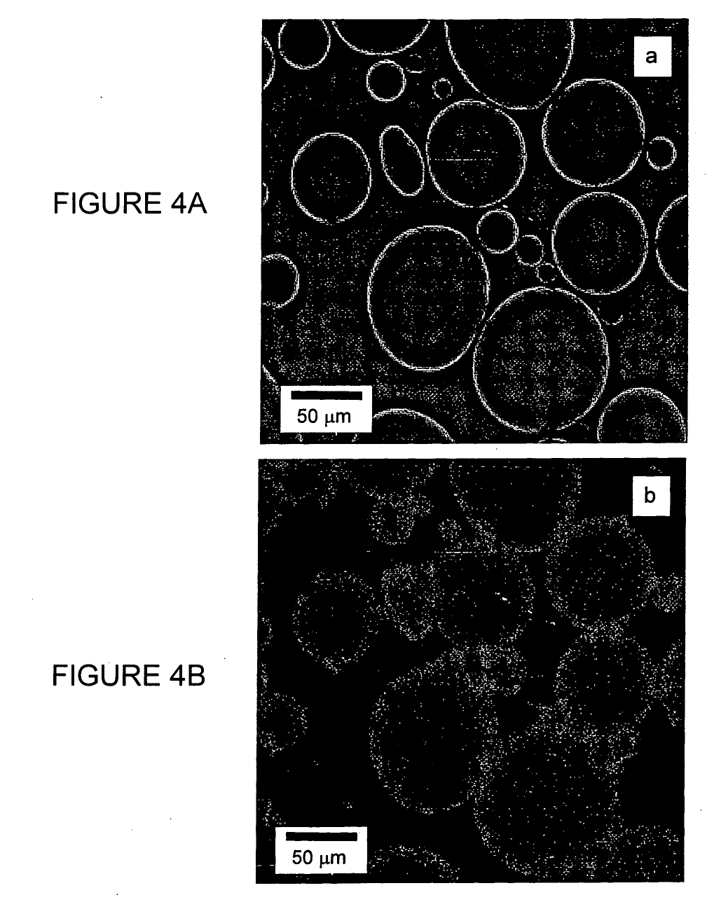

Fluorescently-labeled P4VP/SiO₂ particles were readily synthesized by copolymerizing 1% 1-pyrenylmethyl methacrylate with 4-vinylpyridine in the presence of 1% EGDMA cross-linker and the aqueous silica sol. Confocal laser microscopy studies of an n-dodecane-in-water emulsion prepared using these fluorescently-labelled P4VP/SiO₂ particles (see FIGS. 4a and 4b) confirmed the presence of a red halo surrounding the emulsion droplets. This indicates that the nanocomposite particles were adsorbed at the oil/water interface. The emulsion droplets in FIGS. 4a and 4b were prepared at pH 8.6 using 1 wt. % fluorescently-labeled, 1 wt. % cross-linked P4VP-SiO₂ nanocomposite particles.

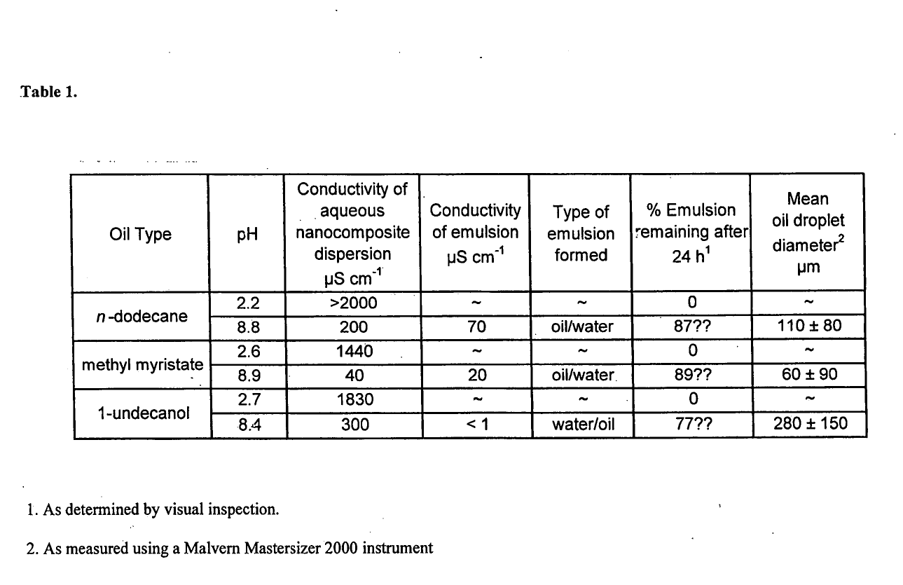

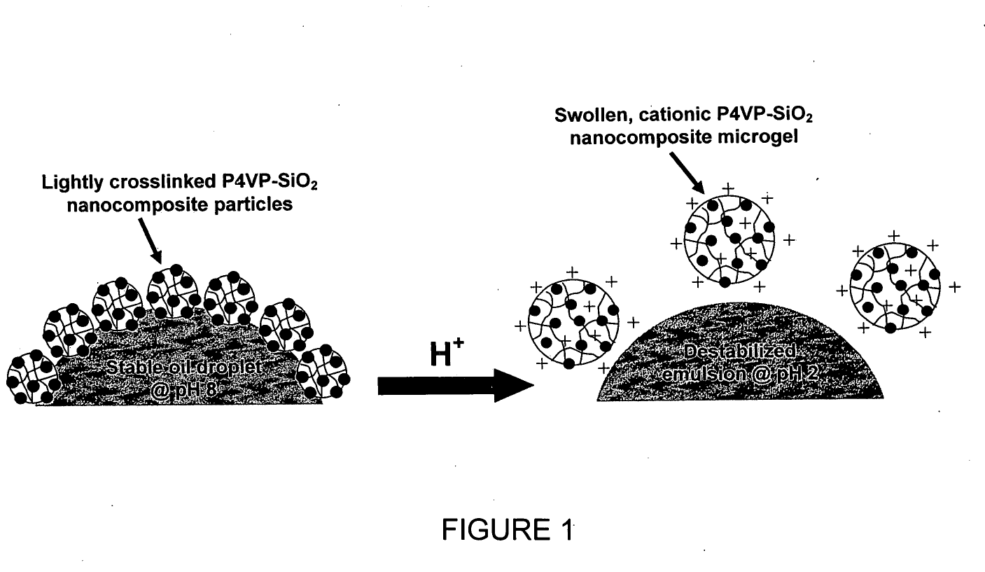

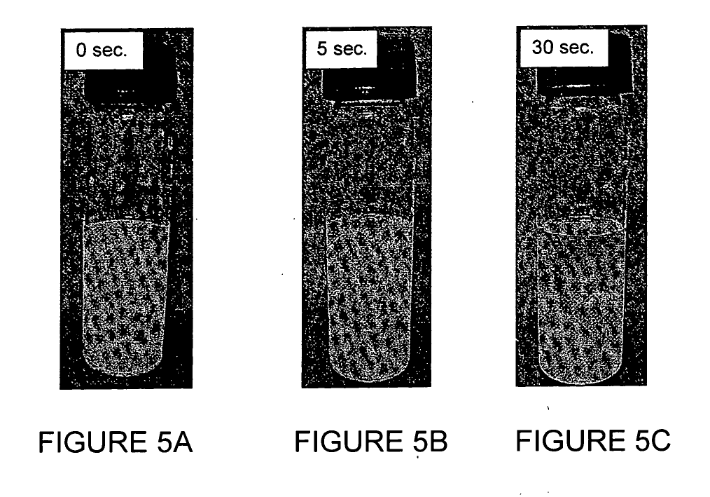

Methyl myristate-in-water and water-in-1-undecanol emulsions were readily demulsified (coalesced) on lowering the solution pH. This is believed to be because the lightly cross-linked nanocomposite particles acquire swollen cationic microgel character as the hydrophobic P4VP surface component becomes hydrophilic on protonation. Hence the particulate emulsifier no longer wets the droplet interface and becomes detached from it, leading to unstable droplets, coalescence and macroscopic phase separation. In the case of methyl myristate, acid-induced demulsification (see FIG. 1) is rapid. The series of digital photographs shown in FIGS. 5a to 5c indicate that gentle agitation of the emulsion is sufficient to cause macroscopic phase separation within thirty seconds of HCl addition at 20° C. The methyl myristate-in-water emulsion of FIGS. 5a to 5c was prepared at pH 8.9 and the pH of the continuous phase was adjusted to pH 2 using aqueous HCl.

Optical microscopy studies recorded during the in situ addition of HCl confirm rapid droplet coalescence. Despite the non-aqueous nature of the continuous phase, rapid demulsification also occurred for acidified water-in-1-undecanol emulsions. Significant droplet coalescence was also observed by optical microscopy for acidified n-dodecane-in-water emulsions, but only partial macroscopic phase separation occurred (even after standing for seven days at ambient temperature).

Comparative experiments using linear (non-crosslinked) poly(4-vinylpyridine)/silica nanocomposite particles as an emulsifier were also carried out. Dissolution at low pH causes a significant increase in solution viscosity, which was detrimental to efficient macroscopic phase separation. This demonstrates that cross-linking leads to a significant enhancement in performance.

### Table 1

Table 1 is a summary of the characterization data obtained for the emulsions prepared by adding 1 wt. % aqueous P4VP-SiO₂ particles (1.0 wt. % crosslinked with ethylene glycol dimethacrylate) to n-dodecane, methyl myristate or 1-undecanol. Equal volumes of n-dodecane and the aqueous nanocomposite dispersion at pH 8-9 were used and emulsification was carried out at 12,000 rpm for 2 min. at 20° C.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

Having thus described the invention, it should be apparent that numerous modifications and adaptations may be resorted to without departing from the scope and fair meaning of the instant invention as set forth hereinabove and as described hereinbelow by the claims.

Although the present invention has been described in considerable detail with reference to certain preferred versions thereof, other versions are possible. Therefore, the spirit and scope of the appended claims should not be limited to the description of the preferred versions described herein.

Any element in a claim that does not explicitly state “means” for performing a specified function or “step” for performing a specified function, should not be interpreted as a “means” or “step” clause as specified in 35 U.S.C. §112.

1. A method of making an emulsion of at least two liquids comprising the step of combining the liquids with a stimulus responsive particulate emulsifier, the emulsifier comprising a nanocomposite microgel, the nanocomposite microgel comprising particles, the particles comprising:
a) an inorganic particulate component; and
b) a cross-linked responsive polymer matrix.

2. The method of claim 1, wherein the nanocomposite microgel has a mean particle size of from about 1×10⁻⁸ nm to about 1×10⁻⁵ nm.

3. The method of claim 1, wherein the nanocomposite microgel comprises a plurality of colloidal particles comprising a plurality of inorganic particles embedded in a cross-linked responsive polymer matrix.

4. The method of claim 3, wherein the nanocomposite microgel has a “currant-bun” morphology wherein inorganic particles are substantially uniformly distributed throughout the colloidal particles of the nanocomposite microgel.

5. The method of claim 3, wherein at least some of the inorganic particles are on the outer surface of the colloidal particles.

6. The method of claim 1, wherein the inorganic particulate component comprises a plurality of particles of an inorganic oxide.

7. The method of claim 6, wherein the inorganic oxide is silica.

8. The method of claim 6, wherein the inorganic oxide comprises silica particles derived from an aqueous silica sol having a mean particle size of from about 1 nm to about 100 nm.

9. The method of claim 1, wherein the volume fraction of inorganic particulate component in the microgel is from about 0.1 to about 0.5.

10. The method of claim 1, wherein the responsive polymer responds to a variation of an external condition by a change in the contact angle of the surface of the particles of the particulate emulsifier with an oil-water interface, measured into the water phase.

11. The method of claim 1, wherein the responsive polymer responds to a variation of an external condition by a change selected from the group consisting of a steric change, a conformational change, a change in protonation, a change in salvation, and a change in hydration.

12. The method of claim 1, wherein the stimulus is at least one of the group consisting of a change in pH, a change in temperature, and a change in ionic strength.

13. The method of claim 1, wherein the responsive polymer matrix comprises a microgel of cross-linked vinyl polymer.

14. The method of claim 13, wherein the vinyl polymer is selected from the group consisting of a polyvinylpyridine and a vinylpyridine copolymer.

15. The method of claim 14, wherein the vinyl polymer is (poly)4-vinylpyridine.

16. The method of claim 13, wherein the inorganic particulate component comprises a plurality of particles of silica; and the particulate emulsifier is a cross-linked vinyl polymer—silica nanocomposite microgel.

17. The method of claim 1, wherein the responsive polymer further comprises a polymer chain; and at least one functional group on the polymer chain, the functional group being selected from the group consisting of acidic and basic functional groups; and wherein the responsive polymer exhibits variable hydrophilicity depending on pH.

18. The method of claim 17, wherein the responsive polymer is responsive within the range of from about pH 2 to about pH 12.

19. The method of claim 1, wherein the responsive polymer has a cross-link density of from about 0.1 mole % to about 5 mole %.

20. The method of claim 1, wherein the responsive polymer is prepared by polymerizing a monomer in the presence of a di-functional monomer as a cross-linking agent.

21. The method of claim 20, wherein the di-functional monomer is ethylene glycol dimethacrylate.

22. An emulsion selected from the group consisting of oil-in-water and water-in-oil emulsions, the emulsion comprising a nanocomposite microgel as a stimulus responsive particulate emulsifier, the nanocomposite microgel comprising particles; wherein the microgel particles comprise: an inorganic particulate component and a cross-linked responsive polymer matrix.

23. The emulsion of claim 22, wherein the stability of the emulsion is dependent upon the response of the responsive polymer to at least one environmental condition.

24. The emulsion of claim 23, wherein application of a stimulus causes breaking of the emulsion.

25. The emulsion of claim 24, wherein the breaking of the emulsion is reversible.

26. The emulsion of claim 23, wherein application of a stimulus causes phase inversion.

27. The emulsion of claim 26, wherein the phase inversion of the emulsion is reversible.

28. The emulsion of claim 23, wherein the particulate emulsifier is a cross-linked vinyl polymer-silica nanocomposite microgel.

29. The emulsion of claim 22, wherein the droplet size of the emulsion is in the range of from about 0.3 to about 100 μm.

30. The emulsion of claim 22, wherein a hydrophilic/hydrophobic balance of the polymer matrix can be varied on application of a stimulus to cause breaking of the emulsion or phase inversion of the emulsion.

31. The emulsion of claim 30, wherein the breaking of the emulsion or the phase inversion of the emulsion is reversible.

32. A method of manufacturing a stimulus responsive oil-in-water or water-in-oil emulsion comprising the step of using a nanocomposite microgel particulate emulsifier, the nanocomposite microgel comprising particles, the particles comprising: an inorganic particulate component; and a cross-linked responsive polymer matrix.

33. A stabilized emulsion comprising a nanocomposite microgel, wherein the microgel particles comprise an inorganic particulate component and a cross-linked responsive polymer matrix, and wherein the stability of the emulsion is dependent on at least one environmental condition.

34. A method of breaking an emulsion, having a nanocomposite microgel as a stimulus responsive particulate emulsifier, wherein the microgel particles comprise an inorganic particulate component and a cross-linked responsive polymer matrix, the method comprising the step of applying a stimulus to cause breaking of the emulsion or phase inversion of the emulsion.

35. The method of breaking an emulsion of claim 34 wherein the step of applying a stimulus further comprises applying a stimulus to vary a hydrophilic/hydrophobic balance of the polymeric matrix to an extent sufficient to cause breaking of the emulsion or phase inversion of the emulsion.

36. The method of breaking an emulsion of claim 34 wherein the step of applying a stimulus further comprises varying at least one environmental condition to an extent sufficient to cause breaking of the emulsion or phase inversion of the emulsion.

37. The method according to claim 36, wherein the environmental condition is selected from the group consisting of temperature, pH, and ionic strength of the emulsion, and wherein the environmental condition is varied to an extent sufficient to break the emulsion.
